(19) United States
(12) Patent Application Publication
(10) Pub. No.: US 2025/0186369 A1
(43) Pub. Date: Jun. 12, 2025

(54) COMPOUNDS, COMPOSITIONS, AND METHODS FOR TREATING, AMELIORATING, AND/OR PRVENTING COCAINE USE DISORDER

(71) Applicant: Drexel University, Philadelphia, PA (US)

(72) Inventors: Sandhya Kortagere, Newtown, PA (US); Rodrigo Espana, Blue Bell, PA (US)

(21) Appl. No.: 18/844,474

(22) PCT Filed: Mar. 6, 2023
(86) PCT No.: PCT/US2023/014589

§ 371 Date: Sep. 6, 2024

Related U.S. Application Data
(60) Provisional application No. 63/317,281, filed on Mar. 7, 2022.

(51) Int. Cl. A61K 31/137; A61K 31/4045; A61K 31/4184; A61K 31/519; A61P 25/30

(52) U.S. Cl. CPC A61K 31/137; A61K 31/4045; A61K 31/4184; A61K 31/519; A61P 25/30

(57) ABSTRACT

The present invention provides a method of treating or ameliorating cocaine use disorder in a subject in need thereof, comprising administering to the subject a compound useful within the disclosure.

### CROSS-REFERENCE TO RELATED APPLICATIONS

This application claims priority to U.S. Provisional Patent Application No. 63/317,281, filed Mar. 7, 2022, entitled “Compounds, Compositions, and Methods for Treating, Ameliorating, and/or Preventing Cocaine Use Disorder”, the disclosure of which is incorporated herein by reference in its entirety.

### BACKGROUND

Relapse to drug taking following periods of withdrawal remains one of the greatest obstacles in treating substance use disorders. In humans, withdrawal from drug use elicits a progressive intensification of drug craving, which is posited to underlie the risk of relapse.

This phenomenon has been modeled preclinically, leading to the hypothesis that withdrawal drives neuroadaptations that underlie intensification of drug craving, including increased motivation for cocaine. Investigation on the neurobiology of cocaine use disorder suggest that adaptations in mesolimbic dopamine (DA) systems may underlie the exaggerated craving and motivation for cocaine observed following periods of withdrawal.

Currently, despite several decades of research there are no FDA-approved pharmacotherapies available to treat, ameliorate, and/or prevent cocaine use disorder. This may be due to the fact that compounds that were developed to block cocaine from binding to DAT have significant psychostimulant effects on their own, and hence are not only ineffective at managing cocaine effects but may produce additive effects.

There is a need in the art for compounds, compositions, and methods for treating and/or ameliorating and/or preventing cocaine use disorder in a subject in need thereof. The present disclosure fulfills this need.

### BRIEF SUMMARY

The disclosure provides a method of preventing, treating, and/or ameliorating cocaine use disorder in a subject in need thereof. In certain embodiments, the method comprises administering to the subject a therapeutically effective amount of at least one dopamine D3 receptor agonist. In certain embodiments, the present disclosure contemplates certain dopamine D3 receptor agonists.

### BRIEF DESCRIPTION OF THE DRAWINGS

For the purpose of illustrating the disclosure, certain embodiments of the present disclosure are depicted in the drawings. However, the disclosure is not limited to the precise arrangements and instrumentalities of the embodiments depicted in the drawings.

FIG. 1 illustrates that SK609 does not promote addiction, at least because that compound does not support self-administration. Shown are injections taken for rats given access to 4 mg/kg SK609 and 0.75 mg/kg cocaine. Dashed line represents acquisition criteria which required 4 consecutive days of 10 injections per session. Cocaine data are aligned to the first day in which rats met acquisition criteria. Data represent mean±s.e.m. *p<0.05, ***p<0.001.

FIGS. 2A-2B illustrate that SK609 reduces motivation for cocaine in a rodent self-administration model. SK609 reduced motivation for cocaine on the Progressive Ratio (PR) schedule. (FIG. 2A) Breakpoints and (FIG. 2B) lever presses following i.p. vehicle, 2 mg/kg or 4 mg/kg SK609. Data are expressed as the mean±s.e.m., percent of baseline responding. *P<0.05.

FIGS. 3A-3C illustrate that SK609 disrupts dopamine (DA) release and uptake and reduces dopamine transporter (DAT) sensitivity to cocaine. (FIG. 3A) DA release, (FIG. 3B) DA uptake (Vₘₐₓ), and (FIG. 3C) DAT sensitivity to cocaine (inhibition of DA uptake; app Kₘ) following vehicle (Veh) or 4 mg/kg SK609. Data are represented as mean s.e.m. *p<0.05, ***p<0.001.

FIGS. 4A-4B illustrate that SK609 reduces expression of pDAT in the striatum of cocaine-treated rats (i.e., expression of pDAT induced by cocaine treatment). (FIG. 4A) Representative western blots for pDAT, tDAT, D3R, βarr2 and GAPDH on striatal synaptosomes derived from rats treated with either saline (sal) or cocaine (CC) (15 mg/kg) or SK609 (4 mg/kg) or Pramipexole (PRX; (S)-N⁶-propyl-4,5,6,7-tetrahydro-1,3-benzothiazole-2,6-diamine) (0.25 mg/kg) with and without cocaine pre-treatment. PRX has been described by others as producing compulsive drug seeking behavior. (FIG. 4B) Densitometry analysis of pDAT expression. Data are presented as mean s.e.m. from 3 independent experiments from n=3 rats/group. *p<0.05, **p<0.001.

FIGS. 5A-5B illustrate that SK609 and its analogues reduce pDAT levels when pretreated with cocaine in a time dependent manner. Time course densitometry analysis of the effects of cocaine (1 μM), PRX (5 nM), SK608 (1 μM), and SK609 (1 μM) on pDAT levels in SH-SY5Y-D3R cells. Different groups of cells were pre-treated with (FIG. 5A) CC, PRX, SK609 and SK608 or (FIG. 5B) CC, CC/PRX, CC/SK609 and CC/SK608 and were collected at the time points indicated. pDAT levels were measured and GAPDH was used as loading control. Data are presented as mean±sd from three independent experiments performed in triplicate. PRX promoted a significant increase in pDAT levels on its own and when combined with CC at all time points compared to SK609 and SK608. SK609 and SK608 treatment post CC significantly reduced pDAT levels. ****p<0.0001 main effect of PRX relative to all other groups.

FIGS. 7A-7D illustrate the finding that pramipexole (PRX), but not SK609, exerts reinforcing effects that promote cocaine acquisition of cocaine self-administration. FIG. 7A: Rats were implanted with jugular catheters and then allowed to self-administer either pramipexole (PRX; 0.25 mg/kg) or SK609 (609; 4 mg/kg) on a 6-hr, fixed ratio 1 schedule of reinforcement (Pre-Cocaine; left gray shading). After 10 days of PRX or SK609 access, rats were switched to cocaine (0.75 mg/kg). Once rats acquired cocaine self-administration behavior (defined as 20 injections of cocaine within a single 6-session) they were allowed another two days of cocaine access (Cocaine; blue shading). Only the last 3 days of cocaine self-administration are plotted. After cocaine self-administration, rats were switched back to PRX and SK609 to assess if these compounds would now maintain reinforced responding (Post-Cocaine; right gray shading). Rats did not acquire PRX or SK609 self-administration, indicating that these compounds are not robust reinforcers. FIG. 7B: Rats displayed higher cumulative intake of PRX compared to SK609 although this effect was not statistically significant. FIG. 7C: Rats that self-administered PRX acquired cocaine self-administration faster than rats that self-administered SK609. FIG. 7D: After cocaine self-administration rats displayed sustained higher overall PRX intake compared to SK609, though again this was not statistically significant. Data are mean±s.e.m. Student's t-test. *p<0.05.

FIGS. 8A-8E illustrate the finding that SK608 and SK609 disrupt dopamine release and uptake and reduce dopamine transporter sensitivity to cocaine. Naive rats were injected intraperitoneally with saline (SAL), pramipexole (PRX; 0.25 mg/kg), SK608 (608; 4 mg/kg), or SK609 (609; 4 mg/kg) 30 min prior to sacrifice. Nucleus accumbens core tissue was then prepared for fast scan cyclic voltammetry and dopamine release and uptake were monitored prior to and after cocaine was bath applied to the tissue. (FIG. 8A) Example dopamine traces, (FIG. 8B) dopamine release and (FIG. 8C) dopamine uptake at baseline, (FIG. 8D) dopamine release and (FIG. 8E) inhibition of dopamine uptake. Data are mean±s.e.m. Student's t-tests or 2-way ANOVAs followed by Holm-Bonferroni tests. *p<0.05, **p<0.01, ***p<0.001. Results show SK608 and SK609 reduce DA release and uptake, while the D2/D3 receptor agonist PRX had no effect on DA release or uptake when compared to Saline group. Interestingly, both SK608 and SK609 significantly reduced DA release and uptake inhibition by cocaine and PRX had no effect.

FIG. 9 illustrates certain functional screening results as described elsewhere herein.

FIG. 10 illustrates certain functional screening results as described elsewhere herein.

### DETAILED DESCRIPTION

The present disclosure relates in one aspect to the unexpected identification of compounds that are useful to prevent, treat and/or ameliorate cocaine use disorder. In certain embodiments, the compounds useful within the present disclosure are dopamine D3 receptor (D3R) agonists. In certain embodiments, the compounds useful within the present disclosure indirectly target dopamine transporters (DATs), activating D3Rs and reducing expression levels and duration of expression of phosphorylated DAT (pDAT) on the membrane. In certain embodiments, the compounds useful within the present disclosure reduce availability of pDAT for cocaine binding. As demonstrated herein, the compounds useful within the present disclosure can reverse the maladaptation of DAT by cocaine using in vitro, ex vivo, and in vivo studies.

Dopamine receptors have attracted attention from biologists and pharmacologists. In the central nervous system, dopamine receptors are widely expressed and involved in the control of locomotion, cognition, emotion and neuroendocrine secretion. In the peripheral system, dopamine receptors are present more prominently in kidney, vasculature and pituitary, where they affect mainly sodium homeostasis, vascular tone, and hormone secretion. While there are numerous examples of functionally-selective ligands that activates one signaling cascade preferentially over others, functionally-selective ligands that alter receptor signaling properties are rare and have not been described for dopamine receptors.

The neurotransmitter dopamine controls a wide variety of physiological and behavioral functions in mammals via five major subtypes of dopamine receptors. They are broadly classified into the “D₁-like” and “D₂-like” dopamine receptors based on pharmacology and function. The D₁-like receptors consist of D₁ and D₅ receptors, while the D₂-like receptors consist of D₂, D₃ and D₄ receptors.

The D₃ receptor primarily couples to the pertussis toxin-sensitive Gₐ-proteins (Gᵢ/Gₒ). When transfected into different cell lines, the D₃ receptor couples to adenylyl cyclase V isoform and initiates signaling events including phosphorylation of mitogen-activated protein (MAP) kinases. D₂ and D₃ dopamine receptors also modulate potassium and calcium channel function. Transfected D₃ receptors couple robustly to natively expressed G-protein coupled inward rectifier potassium (GIRK) and voltage-gated P/Q type calcium channels, and inhibit firing of spontaneous action potentials and secretory activity in the AtT-20 neuroendocrine cell line. The D₃ receptor further couples to natively expressed adenylyl cyclase V, MAP kinases, and ion channels in AtT-20 cells.

There is strong evidence for DAT adaptations in the nucleus accumbens (NAc) core that occur after cocaine withdrawal—with increased DA uptake efficiency at baseline and exaggerated DAT sensitivity to cocaine (i.e., increased inhibition of DA uptake by cocaine). These DAT adaptations are observed exclusively in rats that display incubated craving for cocaine, suggesting that the exaggerated craving is associated with alterations in DAT function. In certain non-limiting embodiments, preventing these maladaptive changes in DAT function, during the withdrawal period, is an effective pharmacotherapeutic approach. To achieve this, dopamine D3 receptor (D3R) agonists, such as the compounds recited herein, can be used to reverse cocaine induced DAT adaptations.

As demonstrated herein, the compounds contemplated in the present disclosure are not addictive and do not promote self-administration in rats that are trained to self-administer cocaine. Further, the compounds contemplated in the present disclosure reduce the motivation for cocaine in the rodent self-administration procedure. Further, the compounds contemplated in the present disclosure reduce stimulated DA release, slow DA uptake, and decrease DAT sensitivity to cocaine measured ex vivo using cyclic voltammetry method. Further, the compounds contemplated in the present disclosure reduce the levels of phosphorylated DAT (pDAT or active DAT) levels, which were increased with cocaine treatment, in striatal synaptosomes. Further, the compounds contemplated in the present disclosure reduce pDAT levels in cells that were pretreated with cocaine. These results indicate that the compounds contemplated in the present disclosure can be used to prevent, treat, and/or ameliorate cocaine use disorders.

In certain embodiments, the compound useful within the disclosure is a selective D₃ receptor agonist. In other embodiments, the compound useful within the disclosure is not a selective activator of the dopamine transporter. In yet other embodiments, the compound useful within the disclosure is not a selective inhibitor of the dopamine transporter.

The disclosure and contents of PCT Application No. PCT/US2011/047263, PCT Application Publication No. WO2012/021629, and U.S. Application Publication No. US 2014/0228355 are incorporated herein by reference in their entireties.

The disclosure and contents of PCT Application No. PCT/US2014/062644, PCT Application Publication No. WO2015/066019, and U.S. Application Publication No. US 2016/0256417 are incorporated herein by reference in their entireties.

### Definitions

As used herein, each of the following terms has the meaning associated with it in this section.

Unless defined otherwise, all technical and scientific terms used herein generally have the same meaning as commonly understood by one of ordinary skill in the art to which this disclosure belongs. Generally, the nomenclature used herein and the laboratory procedures in animal physiology, pharmacology, organic chemistry, and peptide chemistry are those well known and commonly employed in the art.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C≡CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxv, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” or “aralkyl” refers to a functional group wherein a one to six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one to three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, “cocaine use disorder” refers to the reduced ability and/or inability of a cocaine user to discontinue and/or reduce their regular and/or episodic use of cocaine. In certain embodiments, the disorder includes a craving for cocaine use. In certain embodiments, the disorder includes inability to avoid cocaine use upon onset of drug withdrawal symptoms. In certain embodiments, the disorder includes a progressive intensification of drug craving following periods of withdrawal from drug use. In certain embodiments, the disorder includes relapse to drug taking following periods of withdrawal. In certain embodiments, the disorder includes increased cocaine intake over time.

The term “container” includes any receptacle for holding the pharmaceutical composition. For example, In certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “DA” refers to dopamine.

As used herein, the term “DAT” refers to dopamine transporter.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “SK608” or “SK-608” refers to 4-(4-chlorophenyl)-butan-2-amine, and/or a salt or solvate thereof.

As used herein, the term “SK609” or “SK-609” refers to 4-(2-chlorophenyl)-butan-2-amine, and/or a salt or solvate thereof.

As used herein, the term “halide” refers to a halogen atom bearing a negative charge. The halide anions are fluoride (F⁻), chloride (Cl⁻), bromide (Br⁻), and iodide (I⁻).

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quatemized. Up to two heteroatoms may be placed consecutively. Examples include —CH═CHO—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quatemized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

“Instructional material” as that term is used herein includes a publication, a recording, a diagram, or any other medium of expression that may be used to communicate the usefulness of the compounds of the present disclosure. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds of the present disclosure or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

The term “monovalent” as used herein refers to a substituent connecting via a single bond to a substituted molecule. When a substituent is monovalent, such as, for example, F or C₁, it is bonded to the atom it is substituting by a single bond.

As used herein, the term “patient,” “individual” or “subject” refers to a human or a non-human mammal. Non-human mammals include, for example, livestock and pets, such as ovine, bovine, porcine, canine, feline and murine mammals. In certain embodiments, the patient, individual or subject is human.

As used herein, the term “polypeptide” refers to a polymer composed of amino acid residues, related naturally occurring structural variants, and synthetic non-naturally occurring analogs thereof linked via peptide bonds. Synthetic polypeptides may be synthesized, for example, using an automated polypeptide synthesizer. As used herein, the term “protein” typically refers to large polypeptides. As used herein, the term “peptide” typically refers to short polypeptides. Conventional notation is used herein to represent polypeptide sequences: the left-hand end of a polypeptide sequence is the amino-terminus, and the right-hand end of a polypeptide sequence is the carboxyl-terminus.

As used herein, amino acids are represented by the full name thereof, by the three letter code corresponding thereto, or by the one-letter code corresponding thereto, as indicated below: Aspartic Acid, Asp, D; Glutamic Acid, Glu, E; Lysine, Lys, K; Arginine, Arg, R; Histidine, His, H; Tyrosine, Tyr, Y; Cysteine, Cys, C; Asparagine, Asn, N; Glutamine, Gln, Q; Serine, Ser, S; Threonine, Thr, T; Glycine, Gly, G; Alanine, Ala, A; Valine, Val, V; Leucine, Leu, L; Isoleucine, Ile, I; Methionine, Met, M; Proline, Pro, P; Phenylalanine, Phe, F; Tryptophan, Trp, W.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the phrase “pharmaceutically acceptable salt” refers to a salt of the administered compounds prepared from pharmaceutically acceptable non-toxic acids, including inorganic acids, organic acids, solvates, hydrates, or clathrates thereof. Pharmaceutically unacceptable salts may nonetheless possess properties such as high crystallinity, which have utility in the practice of the present disclosure, such as for example utility in process of synthesis, purification or formulation of compounds useful within the methods of the present disclosure. Salts may be comprised of a fraction of one, one or more than one molar equivalent of acid or base with respect to any compound of the present disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluene sulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate).

Suitable pharmaceutically acceptable base addition salts of compounds of the present disclosure include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methyl glucamine) and procaine. All of these salts may be prepared from the corresponding compound by reacting, for example, the appropriate acid or base with the compound.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

As used herein, a “pharmaceutically effective amount,” “therapeutically effective amount,” or “effective amount” of a compound is that amount of compound that is sufficient to provide a beneficial effect to the subject to which the compound is administered.

As used herein, the term “prevent” or “prevention” means no disorder or disease development if none had occurred, and/or no further disorder or disease development if there had already been development of the disorder or disease. Also considered is the ability of one to prevent some or all of the symptoms associated with the disorder or disease.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

As used herein, the term “substituted alkyl,” “substituted cycloalkyl,” “substituted alkenyl,” or “substituted alkynyl” refers to alkyl, cycloalkyl, alkenyl or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

In certain embodiments, each occurrence of alkyl or cycloalkyl is independently optionally substituted with at least one substituent selected from the group consisting of C₁-C₆ alkyl, halo, —OR, phenyl (thus yielding, in non-limiting examples, optionally substituted phenyl-(C₁-C₃ alkyl), such as, but not limited to, benzyl or substituted benzyl) and —N(R)(R), wherein each occurrence of R is independently H, C₁-C₆ alkyl or C₃-C₈ cycloalkyl. In other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N(R)(R), acyl, and C₁-C₆ alkoxycarbonyl, wherein each occurrence of R is independently H, C₁-C₆ alkyl or C₃-C₈ cycloalkyl. In yet other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), and C₁-C₆ alkoxycarbonyl, wherein each occurrence of R is independently H, C₁-C₆ alkyl or C₃-C₈ cycloalkyl.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual sub-combination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

Throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### Compounds

The compounds useful within the disclosure may be synthesized using techniques well-known in the art of organic synthesis.

In one aspect, the compounds useful within the disclosure are selective D₃ receptor agonists. In another aspect, the compounds useful within the disclosure are not activators of the dopamine transporter. In yet another aspect, the compounds useful within the disclosure are not an inhibitor of the dopamine transporter.

In another aspect, the compound is a compound of formula (I):

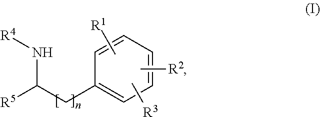

wherein:
R¹, R² and R³ are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and,
n is 2, 3, 4 or 5; or a pharmaceutically acceptable salt or solvate thereof.

In certain embodiments, R¹ is ortho to the

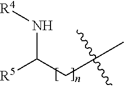

group. In certain embodiments, R¹ is meta to the

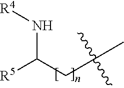

group. In certain embodiments, R¹ is para to the

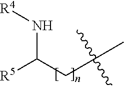

group. In certain embodiments, R¹ is H. In certain embodiments, R¹ is cyano. In certain embodiments, R¹ is hydroxyl. In certain embodiments, R¹ is amino. In certain embodiments, R¹ is acetamido. In certain embodiments, R¹ is halogen. In certain embodiments, R¹ is alkoxy. In certain embodiments, R¹ is nitro. In certain embodiments, R¹ is C₁₋₆ alkyl. In certain embodiments, R¹ is substituted C₁₋₆ alkyl. In certain embodiments, R¹ is heteroalkyl. In certain embodiments, R¹ is heterocyclyl. In certain embodiments, R¹ is substituted heterocyclyl. In certain embodiments, R¹ is aryl. In certain embodiments, R¹ is substituted aryl. In certain embodiments, R¹ is aryl-(C₁₋₃)alkyl. In certain embodiments, R¹ is substituted aryl-(C₁₋₃)alkyl. In certain embodiments, R¹ is carboxy. In certain embodiments, R¹ is alkylcarboxy. In certain embodiments, R¹ is formyl. In certain embodiments, R¹ is alkyl-carbonyl. In certain embodiments, R¹ is aryl-carbonyl. In certain embodiments, R¹ is heteroaryl-carbonyl.

In certain embodiments, R² is ortho to the

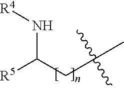

group. In certain embodiments, R² is meta to the

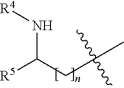

group. In certain embodiments, R² is para to the

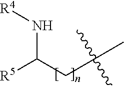

group. In certain embodiments, R² is H. In certain embodiments, R² is cyano. In certain embodiments, R² is hydroxyl. In certain embodiments, R² is amino. In certain embodiments, R² is acetamido. In certain embodiments, R² is halogen. In certain embodiments, R² is alkoxy. In certain embodiments, R² is nitro. In certain embodiments, R² is C₁₋₆ alkyl. In certain embodiments, R² is substituted C₁₋₆ alkyl. In certain embodiments, R² is heteroalkyl. In certain embodiments, R² is heterocyclyl. In certain embodiments, R² is substituted heterocyclyl. In certain embodiments, R² is aryl. In certain embodiments, R² is substituted aryl. In certain embodiments, R² is aryl-(C₁₋₃)alkyl. In certain embodiments, R² is substituted aryl-(C₁₋₃)alkyl. In certain embodiments, R² is carboxy. In certain embodiments, R² is alkylcarboxy. In certain embodiments, R² is formyl. In certain embodiments, R² is alkyl-carbonyl. In certain embodiments, R² is aryl-carbonyl. In certain embodiments, R² is heteroaryl-carbonyl.

In certain embodiments, R³ is ortho to the

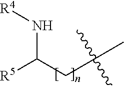

group. In certain embodiments, R³ is meta to the

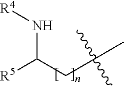

group. In certain embodiments, R³ is para to the

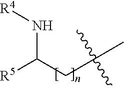

group. In certain embodiments, R³ is H. In certain embodiments, R³ is cyano. In certain embodiments, R³ is hydroxyl. In certain embodiments, R³ is amino. In certain embodiments, R³ is acetamido. In certain embodiments, R³ is halogen. In certain embodiments, R³ is alkoxy. In certain embodiments, R³ is nitro. In certain embodiments, R³ is C₁₋₆ alkyl. In certain embodiments, R³ is substituted C₁₋₆ alkyl. In certain embodiments, R³ is heteroalkyl. In certain embodiments, R³ is heterocyclyl. In certain embodiments, R³ is substituted heterocyclyl. In certain embodiments, R³ is aryl. In certain embodiments, R³ is substituted aryl. In certain embodiments, R³ is aryl-(C₁₋₃)alkyl. In certain embodiments, R³ is substituted aryl-(C₁₋₃)alkyl. In certain embodiments, R³ is carboxy. In certain embodiments, R³ is alkylcarboxy. In certain embodiments, R³ is formyl. In certain embodiments, R³ is alkyl-carbonyl. In certain embodiments, R³ is aryl-carbonyl. In certain embodiments, R³ is heteroaryl-carbonyl.

In certain embodiments, R⁴ is H. In certain embodiments, R⁴ is C₁₋₆ alkyl. In certain embodiments, R⁴ is substituted C₁₋₆ alkyl. In certain embodiments, R⁴ is heteroalkyl. In certain embodiments, R⁴ is heterocyclyl. In certain embodiments, R⁴ is substituted heterocyclyl. In certain embodiments, R⁴ is aryl. In certain embodiments, R⁴ is substituted aryl. In certain embodiments, R⁴ is aryl-(C₁₋₃)alkyl. In certain embodiments, R⁴ is substituted aryl-(C₁₋₃)alkyl.

In certain embodiments, R⁵ is H. In certain embodiments, R⁵ is C₁₋₆ alkyl. In certain embodiments, R⁵ is substituted C₁₋₆ alkyl. In certain embodiments, R⁵ is heteroalkyl. In certain embodiments, R⁵ is heterocyclyl. In certain embodiments, R⁵ is substituted heterocyclyl. In certain embodiments, R⁵ is aryl. In certain embodiments, R⁵ is substituted aryl. In certain embodiments, R⁵ is aryl-(C₁₋₃)alkyl. In certain embodiments, R⁵ is substituted aryl-(C₁₋₃)alkyl.

In certain embodiments, n=1. In certain embodiments, n=2. In certain embodiments, n=3. In certain embodiments, n=4. In certain embodiments, n=5.

In certain embodiments, R¹, R² and R³ are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, carboxy, alkylcarboxy, formyl, alkyl-carbonyl, aryl-carbonyl, and heteroaryl-carbonyl. In other embodiments, R¹, R² and R³ are independently selected from the group consisting of H, cyano, hydroxyl, amino, acetamido, halogen, alkoxy, nitro, C₁₋₆ alkyl, substituted C₁₋₆ alkyl, heteroalkyl, carboxy, alkylcarboxy, formyl, and alkyl-carbonyl. In yet other embodiments, R¹, R² and R³ are independently selected from the group consisting of H, cyano, hydroxyl, amino, acetamido, halogen, alkoxy, nitro, C₁₋₆ alkyl, substituted C₁₋₆ alkyl, heteroalkyl, and carboxy. In yet other embodiments, R¹, R² and R³ are independently selected from the group consisting of H, cyano, hydroxyl, amino, acetamido, halogen, alkoxy, nitro, C₁₋₆ alkyl, and carboxy. In yet other embodiments, R¹, R² and R³ are independently selected from the group consisting of H, cyano, hydroxyl, halogen, alkoxy, nitro, C₁₋₆ alkyl, and carboxy. In yet other embodiments, R¹, R² and R³ are independently selected from the group consisting of H, cyano, halogen, alkoxy, nitro, C₁₋₆ alkyl, and carboxy. In yet other embodiments, R¹ and R² are H, and R³ is chloro. In yet other embodiments, R¹, R² and R³ are either independently or in combination selected from the group consisting of H, fluoro, chloro, bromo, iodo, methoxy, ethoxy, hydroxyl, methyl, ethyl or other lower alkyl or aryl groups.

In certain embodiments, n is 2, 3 or 4. In other embodiments, n is 2 or 3. In yet other embodiments, n is 2.

In certain embodiments, R⁴ and R⁵ are independently selected from the group consisting of H, C₁₋₆ alkyl, substituted C₁₋₆ alkyl, heteroalkyl, heterocyclyl, substituted heterocyclyl, aryl, and substituted aryl. In other embodiments, R⁴ and R⁵ are independently selected from the group consisting of H, C₁₋₆ alkyl, substituted C₁₋₆ alkyl, heteroalkyl, heterocyclyl, and substituted heterocyclyl. In yet other embodiments, R⁴ and R⁵ are independently selected from the group consisting of H, C₁₋₆ alkyl, substituted C₁₋₆ alkyl, and heteroalkyl. In yet other embodiments, R⁴ and R⁵ are independently selected from the group consisting of H, C₁₋₆ alkyl, and substituted C₁₋₆ alkyl. In yet other embodiments, R⁴ and R⁵ are independently selected from the group consisting of H, C₁₋₆ alkyl, and substituted C₁₋₆ alkyl. In yet other embodiments, R⁴ and R⁵ are methyl. In yet other embodiments, R⁵ is H, methyl, ethyl, prop-1-yl, prop-2-yl, hydroxymethyl, 1-hydroxy-ethyl, 2-hydroxy-ethyl, 1-hydroxy-prop-1-yl, 2-hydroxy-prop-1-yl, 3-hydroxy-prop-1-yl, 1-hydroxy-prop-2-yl or 2-hydroxy-prop-2-yl.

In certain embodiments, the compound useful within the disclosure is 2-amino-4-(2-chlorophenyl)butan-1-ol

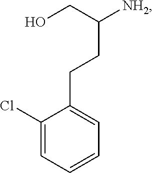 [SMILES: NC(CO)CCc1ccccc1Cl]

or a pharmaceutically acceptable salt or solvate thereof. In certain embodiments, the compound useful within the disclosure is 2-(3-aminohexyl)phenol

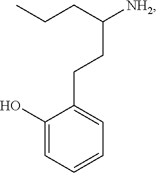

or a pharmaceutically acceptable salt or solvate thereof. In certain embodiments, the compound useful within the disclosure is 4-(2-chlorophenyl)-2-methylamino-butane (also known as 4-(2-chlorophenyl)-N-methyl-butan-2-amine)

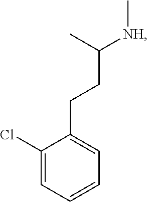

or a pharmaceutically acceptable salt or solvate thereof. In certain embodiments, the compound useful within the disclosure is 4-(2-chlorophenyl)-2-amino-butane (also known as 4-(2-chlorophenyl)-butan-2-amine)

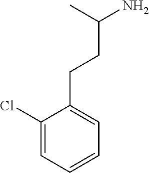

(also known as SK609 or SK-609), or a pharmaceutically acceptable salt or solvate thereof. In certain embodiments, the compound useful within the disclosure is 4-(2-fluorophenyl)butan-2-amine

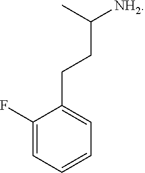

In certain embodiments, the compound useful within the disclosure is 4-(2-bromophenyl)butan-2-amine

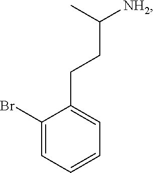

or a pharmaceutically acceptable salt or solvate thereof. In certain embodiments, the compound useful within the disclosure is 4-(2-iodophenyl)butan-2-amine

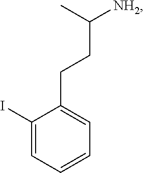

or a pharmaceutically acceptable salt or solvate thereof. In certain embodiments, the compound useful within the disclosure is 4-(2-methoxyphenyl)butan-2-amine

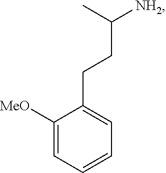

or a pharmaceutically acceptable salt or solvate thereof. In certain embodiments, the compound useful within the disclosure is 2-(3-aminobutyl)phenol

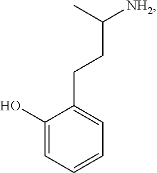

or a pharmaceutically acceptable salt or solvate thereof. In certain embodiments, the compound useful within the disclosure is 3-(3,4-diethoxyphenyl)propan-1-amine

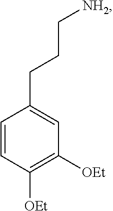 [SMILES: CCOc1ccc(CCCN)cc1OCC]

or a pharmaceutically acceptable salt or solvate thereof. In certain embodiments, the compound useful within the disclosure is 4-(4-chlorophenyl)butan-2-amine

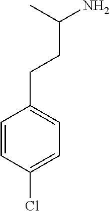

(also known as SK608 or SK-608), or a pharmaceutically acceptable salt or solvate thereof. In certain embodiments, the compound useful within the disclosure is 4-(4-methoxyphenyl)butan-2-amine

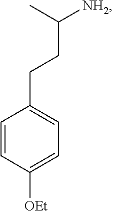

or a pharmaceutically acceptable salt or solvate thereof.

In yet another aspect, the compound is a compound of formula (II):

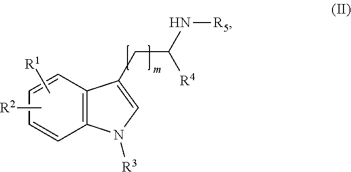

wherein:
R¹ and R² are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R³ and R⁴ are independently selected from the group consisting of H, C₁₋₆ alkyl, and substituted C₁₋₆ alkyl or aryl or heteroaryl;
R⁵ is selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and,
m is 1, 2, or 3;
   or a pharmaceutically acceptable salt or solvate thereof.

In certain embodiments, R¹ is ortho to the indole N atom. In certain embodiments, R¹ is meta to the indole N atom. In certain embodiments, R¹ is para to the indole N atom. In certain embodiments, R¹ is H. In certain embodiments, R¹ is cyano. In certain embodiments, R¹ is hydroxyl. In certain embodiments, R¹ is amino. In certain embodiments, R¹ is acetamido. In certain embodiments, R¹ is halogen. In certain embodiments, R¹ is alkoxy. In certain embodiments, R¹ is nitro. In certain embodiments, R¹ is C₁₋₆ alkyl. In certain embodiments, R¹ is substituted C₁₋₆ alkyl. In certain embodiments, R¹ is heteroalkyl. In certain embodiments, R¹ is heterocyclyl. In certain embodiments, R¹ is substituted heterocyclyl. In certain embodiments, R¹ is aryl. In certain embodiments, R¹ is substituted aryl. In certain embodiments, R¹ is aryl-(C₁₋₃)alkyl. In certain embodiments, R¹ is substituted aryl-(C₁₋₃)alkyl. In certain embodiments, R¹ is carboxy. In certain embodiments, R¹ is alkylcarboxy. In certain embodiments, R¹ is formyl. In certain embodiments, R¹ is alkyl-carbonyl. In certain embodiments, R¹ is aryl-carbonyl. In certain embodiments, R¹ is heteroaryl-carbonyl.

In certain embodiments, R² is ortho to the indole N atom. In certain embodiments, R² is meta to the indole N atom. In certain embodiments, R² is para to the indole N atom. In certain embodiments, R² is H. In certain embodiments, R² is cyano. In certain embodiments, R² is hydroxyl. In certain embodiments, R² is amino. In certain embodiments, R² is acetamido. In certain embodiments, R² is halogen. In certain embodiments, R² is alkoxy. In certain embodiments, R² is nitro. In certain embodiments, R² is C₁₋₆ alkyl. In certain embodiments, R² is substituted C₁₋₆ alkyl. In certain embodiments, R² is heteroalkyl. In certain embodiments, R² is heterocyclyl. In certain embodiments, R² is substituted heterocyclyl. In certain embodiments, R² is aryl. In certain embodiments, R² is substituted aryl. In certain embodiments, R² is aryl-(C₁₋₃)alkyl. In certain embodiments, R² is substituted aryl-(C₁₋₃)alkyl. In certain embodiments, R² is carboxy. In certain embodiments, R² is alkylcarboxy. In certain embodiments, R² is formyl. In certain embodiments, R² is alkyl-carbonyl. In certain embodiments, R² is aryl-carbonyl. In certain embodiments, R² is heteroaryl-carbonyl.

In certain embodiments, R³ is H. In certain embodiments, R³ is C₁₋₆ alkyl. In certain embodiments, R³ is substituted C₁₋₆ alkyl. In certain embodiments, R³ is substituted aryl. In certain embodiments, R³ is substituted heteroaryl.

In certain embodiments, R⁴ is H. In certain embodiments, R⁴ is C₁₋₆ alkyl. In certain embodiments, R⁴ is substituted C₁₋₆ alkyl. In certain embodiments, R⁴ is substituted aryl. In certain embodiments, R⁴ is substituted heteroaryl.

In certain embodiments, R⁵ is H. In certain embodiments, R⁵ is C₁₋₆ alkyl. In certain embodiments, R⁵ is substituted C₁₋₆ alkyl. In certain embodiments, R⁵ is heteroalkyl. In certain embodiments, R⁵ is heterocyclyl. In certain embodiments, R⁵ is substituted heterocyclyl. In certain embodiments, R⁵ is aryl. In certain embodiments, R⁵ is substituted aryl. In certain embodiments, R⁵ is aryl-(C₁₋₃)alkyl. In certain embodiments, R⁵ is substituted aryl-(C₁₋₃)alkyl.

In certain embodiments, m=1. In certain embodiments, m=2. In certain embodiments, m=3.

In certain embodiments, R¹ and R² are independently selected from the group consisting of H, cyano, hydroxyl, amino, acetamido, halogen, alkoxy, nitro, C₁₋₆ alkyl, substituted C₁₋₆ alkyl, carboxy, alkylcarboxy, formyl, alkyl-carbonyl, aryl-carbonyl, and heteroaryl-carbonyl. In other embodiments, R¹ and R² are independently selected from the group consisting of H, cyano, hydroxyl, halogen, and alkoxy, C₁₋₆ alkyl, and substituted C₁₋₆ alkyl.

In certain embodiments, R³ and R⁴ are independently selected from the group consisting of H, and C₁₋₆ alkyl.

In certain embodiments, R⁵ is selected from the group consisting of H, C₁₋₆ alkyl, and substituted C₁₋₆ alkyl.

In certain embodiments, m is 1, or 2.

In certain embodiments, the compound useful within the disclosure is 2-(5-chloro-1-methyl-1H-indol-3-yl)ethanamine

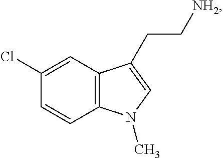

or a pharmaceutically acceptable salt or solvate thereof. In certain embodiments, the compound useful within the disclosure is 1-(5-fluoro-1-methyl-1H-indol-3-yl)propan-2-amine

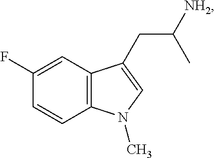

or a pharmaceutically acceptable salt or solvate thereof. In certain embodiments, the compound useful within the disclosure is 1-(5-methoxy-1-methyl-1H-indol-3-yl)propan-2-amine

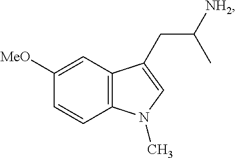 [SMILES: COc1ccc2c(c1)c(CC(C)N)cn2C]

or a pharmaceutically acceptable salt or solvate thereof.

In yet another aspect, the compound useful within the disclosure is 2,7-diamino-5-(4-fluorophenyl)-4-oxo-3,4,5,6-tetrahydropyrido[2,3-d]pyrimidine-6-carbonitrile

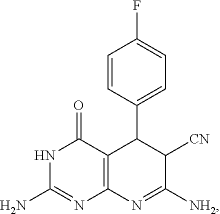 [SMILES: N#CC1C(N)=Nc2nc(N)[nH]c(=O)c2C1c1ccc(F)cc1]

or a pharmaceutically acceptable salt or solvate thereof.

In yet another aspect, the compound useful within the disclosure is (Z)-2-(1H-benzo[d]imidazol-2-yl)-N′-hydroxy-3-(4-methoxyphenyl)propanimidamide

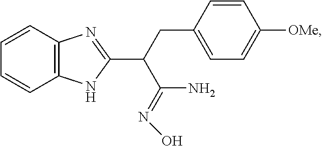 [SMILES: COc1ccc(CC(/C(N)=N/O)c2nc3ccccc3[nH]2)cc1]

or a pharmaceutically acceptable salt or solvate thereof.

The compounds of the disclosure may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H. ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods

In certain embodiments, the disclosure provides a method of preventing, treating and/or ameliorating cocaine use disorder in a subject in need thereof. In certain embodiments, the method comprises administering to the subject a therapeutically effective amount of at least one dopamine D3 receptor agonist. In certain embodiments, the agonist is any of the compounds described elsewhere herein. In certain embodiments, the agonist is not addictive. In certain embodiments, administration of the agonist reduces and/or eliminates the subject's motivation for cocaine. In certain embodiments, the method comprises administering to the subject a pharmaceutical composition comprising a therapeutically effective amount of at least one compound of the present disclosure.

In certain embodiments, the compound useful within the disclosure is selected from the group consisting of: 2-amino-4-(2-chlorophenyl)butan-1-ol; 2-(3-aminohexyl) phenol; 4-(2-chlorophenyl)-2-methylamino-butane (also known as 4-(2-chlorophenyl)-N-methylbutan-2-amine); 4-(2-chlorophenyl)-2-amino-butane; 4-(2-fluorophenyl)butan-2-amine; 4-(2-bromophenyl)butan-2-amine; 4-(2-iodophenyl)butan-2-amine; 4-(2-methoxyphenyl)butan-2-amine; 2-(3-aminobutyl) phenol; 3-(3,4-diethoxyphenyl)propan-1-amine; 4-(4-chlorophenyl)butan-2-amine; 4-(4-methoxyphenyl)butan-2-amine; 2-(5-chloro-1-methyl-1H-indol-3-yl)ethanamine; 1-(5-fluoro-1-methyl-1H-indol-3-yl)propan-2-amine; 1-(5-methoxy-1-methyl-1H-indol-3-yl)propan-2-amine; 2,7-diamino-5-(4-fluorophenyl)-4-oxo-3,4,5,6-tetrahydropyrido[2,3-d]pyrimidine-6-carbonitrile; (Z)-2-(1H-benzo[d]imidazol-2-yl)-N′-hydroxy-3-(4-methoxyphenyl) propanimidamide; mixtures thereof, or a pharmaceutically acceptable salt or solvate thereof.

In certain embodiments, the subject is a mammal, such as but not limited to a human.

### Combination Therapies

The compounds of the present invention are intended to be useful in the methods of present invention in combination with one or more additional compounds useful for treating, ameliorate, and/or prevent any of the diseases or disorders contemplated within the invention. These additional compounds may comprise compounds of the present invention or compounds, e.g., commercially available compounds, known to treat, prevent, or reduce any of the symptoms of the diseases or disorders contemplated within the invention.

In non-limiting examples, the compounds of the present invention may be used in combination with one or more of the following drugs: long-acting amphetamine formulations, modafinil, topiramate, doxazosin, and combined topiramate and mixed amphetamine salts extended-release (MAS-ER), hypocretin receptor antagonists like SB334867 (also known as N-(2-Methyl-6-benzoxazolyl)-N′-1,5-naphthyridin-4-yl-urea), suvorexant, almorexant, RTIOX-276 (also known as 2-(1-(3,4-dimethoxybenzyl)-6-methoxy-7-(2,2,2-trifluoroethoxy)-3,4-dihydroisoquinolin-2(1H)-yl)-N-(pyridin-3-ylmethyl)acetamide), and so forth.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions useful within the present disclosure to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the present disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the present disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the present disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions useful within the disclosure are formulated using one or more pharmaceutically acceptable excipients or carriers. In certain embodiments, the pharmaceutical compositions of the present disclosure comprise a therapeutically effective amount of at least one compound useful within the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions useful within the disclosure are administered to the patient in dosages that range from one to five times per day or more. In other embodiments, the compositions useful within the disclosure are administered to the patient in range of dosages that include, but are not limited to, once every day, every two, days, every three days to once a week, and once every two weeks. It will be readily apparent to one skilled in the art that the frequency of administration of the various combination compositions useful within the disclosure will vary from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

Compounds for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound is from about 1 mg and about 2,500 mg. In some embodiments, a dose of a compound of the present disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating a disease or disorder) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the present disclosure, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the present disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY—P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds of the present disclosure, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Parenteral Administration

For parenteral administration, the compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the present disclosure may be administered in the form of microparticles, for example, by injection or in the form of wafers or discs by implantation.

In certain embodiments of the present disclosure, the compounds of the present disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound will depend on the age, sex and weight of the patient, the current medical condition of the patient and the progression of the disease or disorder in the patient being treated. The skilled artisan will be able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the present disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Example 1: Compounds of the Disclosure do not Promote Addiction

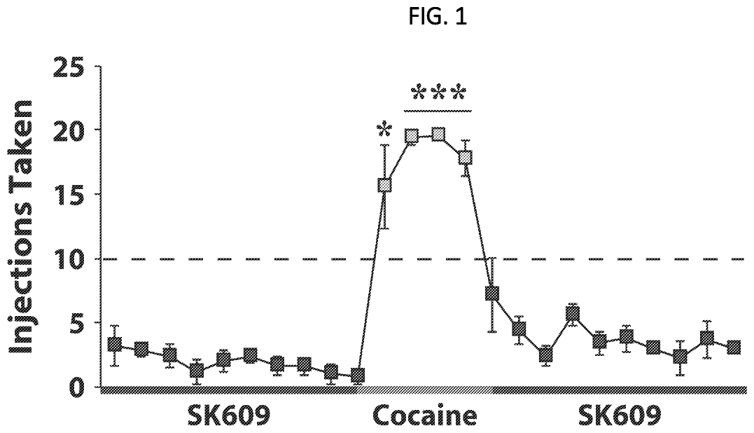

Shown in FIG. 1 are injections taken for rats given access to 4 mg/kg SK609 and 0.75 mg/kg cocaine. Dashed line represents acquisition criteria which required 4 consecutive days of 10 injections per session. Cocaine data are aligned to the first day in which rats met acquisition criteria. Data represent mean s.e.m. *p<0.05, ***p<0.001.

### Example 2: Compounds of the Disclosure Reduce Motivation for Cocaine in a Rodent Self-Administration Model

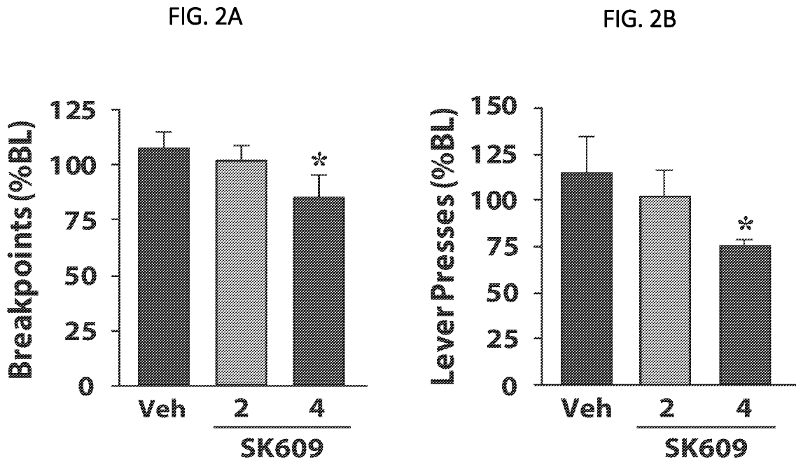

FIGS. 2A-2B illustrate that exemplary compound SK609 reduced motivation for cocaine on the PR schedule. (FIG. 2A) Breakpoints and (FIG. 2B) lever presses following i.p. vehicle, 2 mg/kg or 4 mg/kg SK609. Data are expressed as the mean±s.e.m., percent of baseline responding. *P<0.05.

### Example 3: Compounds of the Disclosure Disrupt Dopamine (DA) Release and Uptake and Reduces Dopamine Transporter (DAT) Sensitivity to Cocaine

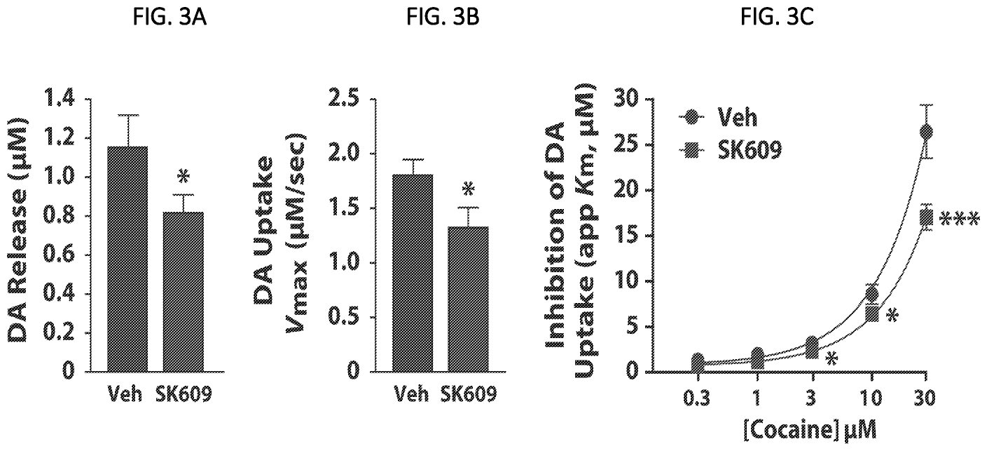

FIGS. 3A-3C illustrate that SK609 disrupts dopamine (DA) release and uptake and reduces dopamine transporter (DAT) sensitivity to cocaine. (FIG. 3A) DA release, (FIG. 3B) DA uptake (Vₘₐₓ), and (FIG. 3C) DAT sensitivity to cocaine (inhibition of DA uptake; app Kₘ) following vehicle (Veh) or 4 mg/kg SK609. Data are represented as mean±s.e.m. *p<0.05, ***p<0.001.

### Example 4: Compounds of the Disclosure Reduce Expression of pDAT Induced by Cocaine Treatment

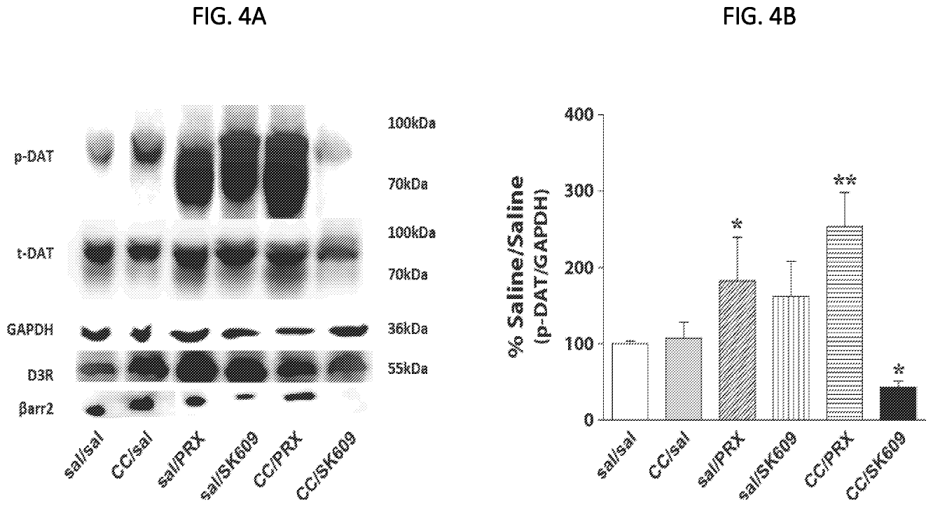

FIGS. 4A-4B illustrate that SK609 reduces expression of pDAT in the striatum of cocaine-treated rats. (FIG. 4A) Representative western blots for pDAT, tDAT, D3βarr2 and GAPDH on striatal synaptosomes derived from rats treated with either saline (sal) or cocaine (CC) (15 mg/kg) or SK609 (4 mg/kg) or Pramipexole (PRX) (0.25 mg/kg) with and without cocaine pre-treatment. (FIG. 4B) Densitometry analysis of pDAT expression. Data are presented as mean±s.e.m. from 3 independent experiments from n=3 rats/group. *p<0.05, **p<0.001.

### Example 5: Compounds of the Disclosure Reduce pDAT Levels when Pretreated with Cocaine in a Time Dependent Manner

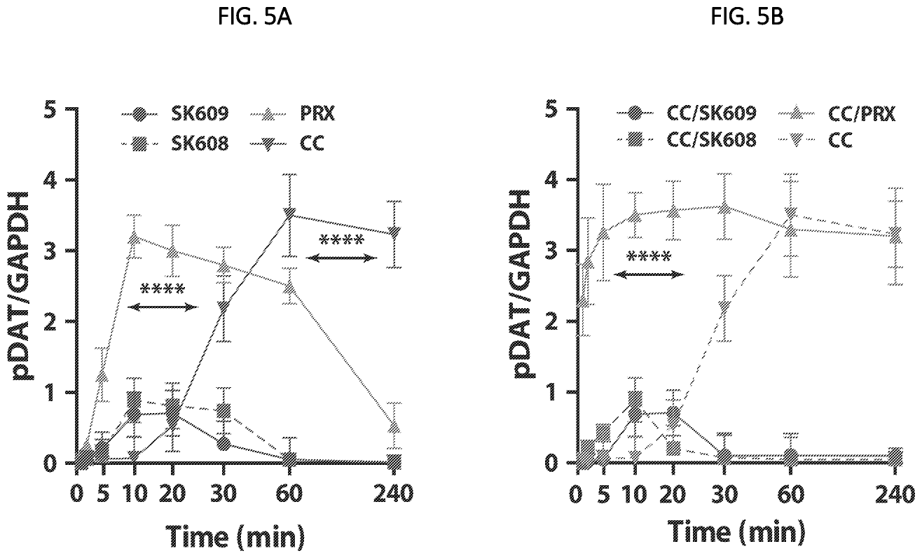

FIGS. 5A-5B illustrate that SK609 and its analogues reduce pDAT levels when pretreated with cocaine in a time dependent manner. Time course densitometry analysis of the effects of cocaine (1 μM), PRX (5 nM), SK608 (1 μM), and SK609 (1 μM) on pDAT levels in SH-SY5Y-D3R cells. Different groups of cells were pre-treated with (FIG. 5A) CC, PRX, SK609 and SK608 or (FIG. 5B) CC, CC/PRX, CC/SK609 and CC/SK608 and were collected at the time points indicated. pDAT levels were measured and GAPDH was used as loading control. Data are presented as mean±sd from three independent experiments performed in triplicate. PRX promoted a significant increase in pDAT levels on its own and when combined with CC at all time points compared to SK609 and SK608. SK609 and SK608 treatment post CC significantly reduced pDAT levels. ****p<0.0001 main effect of PRX relative to all other groups.

### Example 6: Pramipexole (PRX), but not Compounds of the Disclosure, Exerts Reinforcing Effects that Promote Cocaine Acquisition of Cocaine Self-Administration

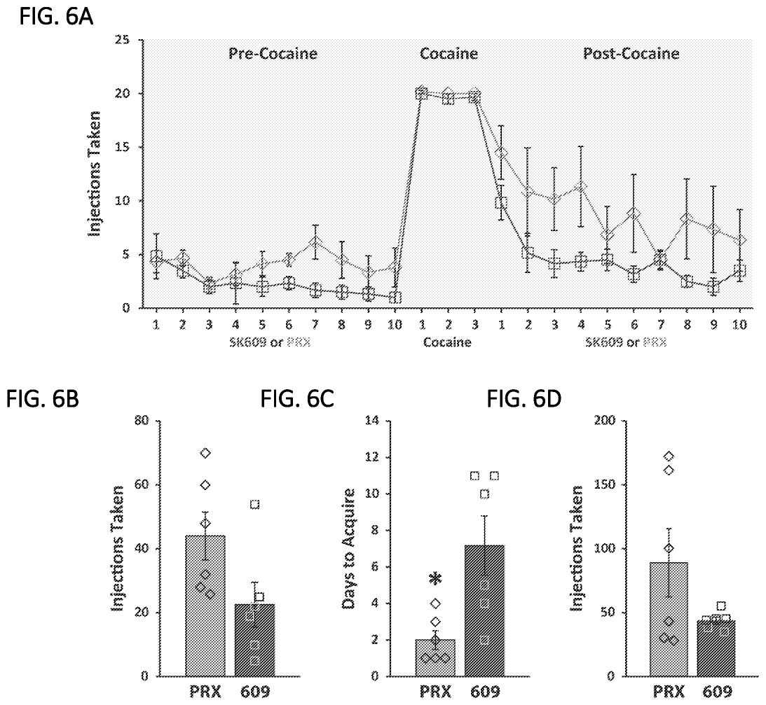

As illustrated in FIG. 7A, rats were implanted with jugular catheters and then allowed to self-administer either pramipexole (PRX; 0.25 mg/kg) or SK609 (609; 4 mg/kg) on a 6-hr, fixed ratio 1 schedule of reinforcement (Pre-Cocaine; left gray shading). After 10 days of PRX or SK609 access, rats were switched to cocaine (0.75 mg/kg). Once rats acquired cocaine self-administration behavior (defined as 20 injections of cocaine within a single 6-session) they were allowed another two days of cocaine access (Cocaine; blue shading). Only the last 3 days of cocaine self-administration are plotted. After cocaine self-administration, rats were switched back to PRX and SK609 to assess if these compounds would now maintain reinforced responding (Post-Cocaine; right gray shading). Rats did not acquire PRX or SK609 self-administration, indicating that these compounds are not robust reinforcers.

As illustrated in FIG. 7B, rats displayed higher cumulative intake of PRX compared to SK609 although this effect was not statistically significant.

As illustrated in FIG. 7C, rats that self-administered PRX acquired cocaine self-administration faster than rats that self-administered SK609.

As illustrated in FIG. 7D, after cocaine self-administration rats displayed sustained higher overall PRX intake compared to SK609, though again this was not statistically significant. Data are mean±s.e.m. Student's t-test. *p<0.05.

### Example 7: SK608 and SK609 Disrupt Dopamine Release and Uptake and Reduce Dopamine Transporter Sensitivity to Cocaine

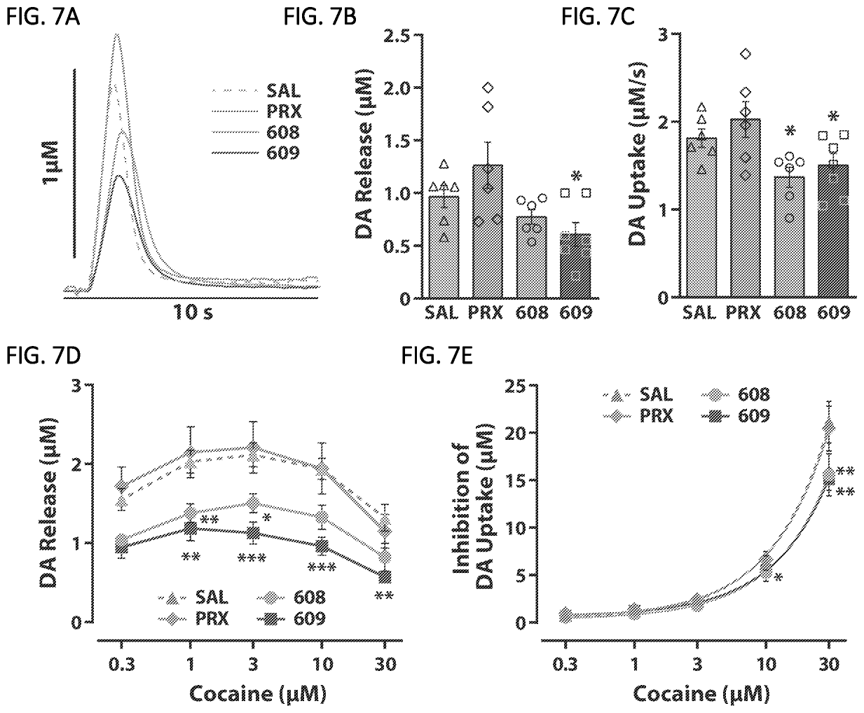

Naive rats were injected intraperitoneally with saline (SAL), pramipexole (PRX; 0.25 mg/kg), SK608 (608; 4 mg/kg), or SK609 (609; 4 mg/kg) 30 min prior to sacrifice. Nucleus accumbens core tissue was then prepared for fast scan cyclic voltammetry and dopamine release and uptake were monitored prior to and after cocaine was bath applied to the tissue. (FIG. 8A) Example dopamine traces, (FIG. 8B) dopamine release and (FIG. 8C) dopamine uptake at baseline, (FIG. 8D) dopamine release and (FIG. 8E) inhibition of dopamine uptake. Data are mean s.e.m. Student's t-tests or 2-way ANOVAs followed by Holm-Bonferroni tests. *p<0.05, **p<0.01, ***p<0.001.

As demonstrated herein, SK608 and SK609 reduce DA release and uptake, while the D2/D3 receptor agonist PRX had no effect on DA release or uptake when compared to Saline group. Interestingly, both SK608 and SK609 significantly reduced DA release and uptake inhibition by cocaine and PRX had no effect.

### Example 8

Selected data provided herein demonstrate that compounds of the disclosure selectively activate the D₃ receptor (and not the D₂ receptor). Compounds of the disclosure are atypical D₃ receptor agonists, because they bind to the D₃ receptor but completely abolish the receptor's tolerance and slow response termination properties. Compounds of the disclosure pharmacologically convert the D₃ receptor into the functional equivalent of a D₂ receptor.

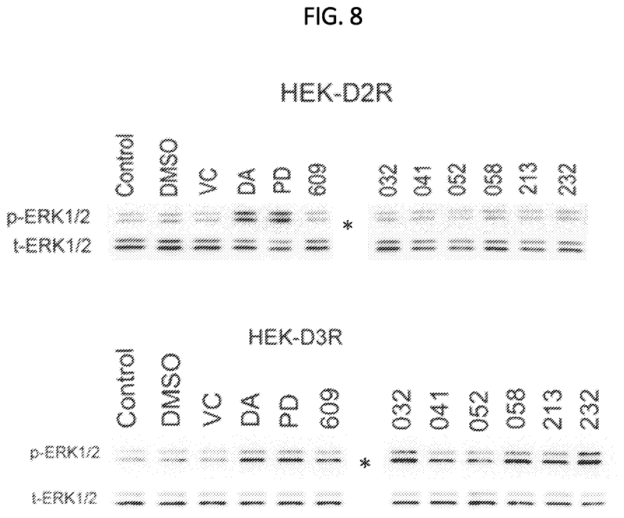

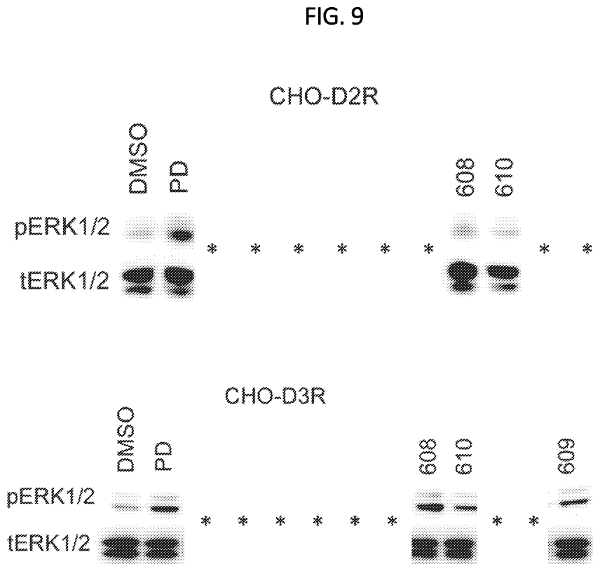

Selected compounds of the disclosure, as well as dopamine (DA) and PD128907 [a commercially available dopamine D₂/D₃ agonist; (4aR,10bR)-3,4a,4,10b-tetrahydro-4-propyl-2H,5H-[1]benzopyrano-[4,3-b]-1,4-oxazin-9-ol], were tested in an established ERK1/2 MAPK activation assay for dopamine D₂ or D₃ agonist activity. The results of these side-by-side tests, comprising immunoblot analysis of phosphorylated ERKT/2 MAPK, are illustrated in FIGS. 9-10.

Functional screening was conducted with vehicle or test compounds using ERK1/2 phosphorylation as endpoint as described herein. 0.4×10⁶ CHO-D₃R (or HEK-D₃R) and CHO-D₂R (or HEK-D₂R) cells were seeded in 6-well plates and cultured in 3 ml DMEM complete growth medium for 48 h. Cells were washed with 1 ml PBS and incubated with 3 ml pre-warmed DMEM without serum for 2 h. Reference compound dopamine (noted as DA) (1 μM with 10 μg/ml ascorbic acid), D₂-like agonist PD128907 (1 μM in 0.01% DMSO) or compounds of the disclosure (1 μM in 0.01% DMSO) were added to the cells and incubated for indicated time (2 min) at 37° C. The reaction was stopped by aspirating the medium and washing the cells with 1 ml PBS followed by the addition of 100 μl/well lysis buffer with 1× Protease Inhibitor Cocktail and 1× Phosphatase Inhibitor Cocktail and 1 mM PMSF to solubilize cells.

Cell lysates were assayed or protein content with DC Protein Assay Kit II (Bio-Rad). The protein samples were treated with loading buffer, boiled for 5 min at 100° C. in a water bath, spinned quickly at 2,500 g, and then 5 μg total protein/lane were loaded to Novex® 4-20% Tris-Glycine Mini Gel for separation (Invitrogen) for 2 h and transferred to a PVDF membrance (Thermo Scientific). Immunoblotting was performed with rabbit anti-phospho-p44/42 MAPK polyclonal antibody (1:5,000, Cell Signaling Technology) and peroxidase-conjugated goat anti-rabbit Ig G (H+L) (1:5,000, Jackson Immuno Research Laboratories, INC). Total p44/42 MAPK levels in the same blots were also determined with rabbit anti-p44/42 MAPK monoclonal or polyclonal antibody (1:5000, Cell Signaling Technology) after stripping the blots of the same PVDF membrance. Chemiluminescence detection was performed using the SuperSignal West Dura Extended Duration Substrate detection kit (Thermo Scientific, Rockford, IL) and phosphorylated or total ERK1/2 MAPK immunoblots were quantified by densitometry with ImageQuant LAS4000 (GE Healthcare Bio-Sciences, Pittsburgh, PA).

The results are illustrated in FIGS. 9-10. Representative western blots of ERK1/2 phosphorylation induced by activation of D₂ receptor (top panel of each FIG.) or D₃ receptor (bottom panel of each FIG.) by 1 μM of PD128907, DA, or compounds of the disclosure are shown. The positive control PD128609 activated both D₂ and D₃ receptors, while the compounds of the disclosure selectively activated only the D₃ receptor.

The biological results presented herein indicate that the compounds of the disclosure selectively activate the D₃ receptor, and thus have measurable effect on dopaminergic D₂-like receptor mediated actions. It follows that the compounds of the disclosure are D₃ receptor agonists.

TABLE 1

| Compounds | Structure |
|---|---|
| 032 (as HCl salt) | 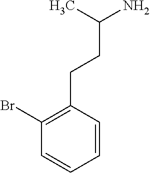 |
| 041 (as HCl salt) | 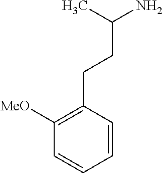 |
| 052 (as HCl salt) | 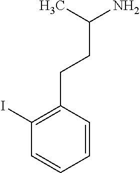 |
| 058 (as free base) | 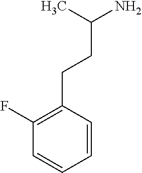 |
| 213 (as HCl salt) | 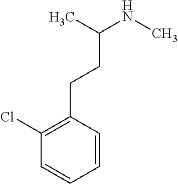 |
| 232 (as HCl salt) | 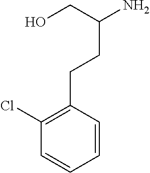 [SMILES: NC(CO)CCc1ccccc1Cl] |
| 608 (as free base) | 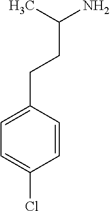 |
| 609 (free base; also as HCl salt) | 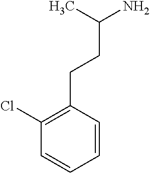 |
| 610 (as free base) | 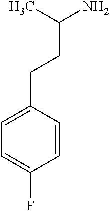 |
| PD128907 (as free base) | 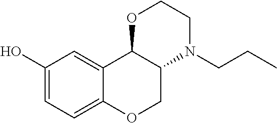 [SMILES: CCCN1CCO[C@@H]2c3cc(O)ccc3OC[C@H]21] |

### Enumerated Embodiments

The following enumerated embodiments are provided, the numbering of which is not to be construed as designating levels of importance:

Embodiment 1: A method of preventing, treating, and/or ameliorating cocaine use disorder in a subject in need thereof, the method comprising administering to the subject a therapeutically effective amount of at least one dopamine D3 receptor agonist.

Embodiment 2: The method of Embodiment 1, wherein the agonist is selected from the group consisting of
a compound of formula (T):

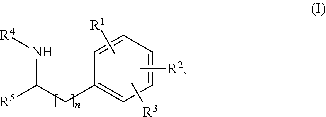

wherein in (I):
   R¹, R² and R³ are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
   R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and,
   n is 2, 3, 4 or 5;
a compound of formula (II):

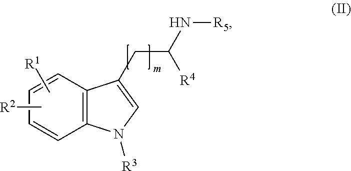

wherein in (II):
   R¹ and R² are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
   R³ and R⁴ are independently selected from the group consisting of H, C₁₋₆ alkyl, and substituted C₁₋₆ alkyl;
   R⁵ is selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and,
   m is 1, 2, or 3;
2,7-diamino-5-(4-fluorophenyl)-4-oxo-3,4,5,6-tetrahydropyrido[2,3-d]pyrimidine-6-carbonitrile;
(Z)-2-(1H-benzo[d]imidazol-2-yl)-N′-hydroxy-3-(4-methoxyphenyl)propanimidamide; a pharmaceutically acceptable salt or solvate thereof, and any mixtures thereof.

Embodiment 3: The method of any one of Embodiments 1-2, wherein the agonist is not addictive to the subject.

Embodiment 4: The method of any one of Embodiments 1-3, wherein administration of the agonist reduces and/or eliminates the subject's motivation for cocaine use.

Embodiment 5: The method of any one of Embodiments 2-4, wherein in (I) R¹, R² and R³ are independently selected from the group consisting of H, cyano, halogen, alkoxy, nitro, C₁₋₆ alkyl, and carboxy.

Embodiment 6: The method of any one of Embodiments 2-5, wherein in (I) R⁴ and R⁵ are independently selected from the group consisting of H, C₁₋₆ alkyl, and substituted C₁₋₆ alkyl.

Embodiment 7: The method of any one of Embodiments 2-6, wherein in (I) n is 2.

Embodiment 8: The method of any one of Embodiments 2-4, wherein in (II) m is 1.

Embodiment 9: The method of any one of Embodiments 2-8, wherein the at least one compound is selected from the group consisting of: 2-amino-4-(2-chlorophenyl)butan-1-ol; 2-(3-aminohexyl)phenol; 4-(2-chlorophenyl)-2-methylamino-butane; 4-(2-chlorophenyl)-2-amino-butane; 4-(2-fluorophenyl)butan-2-amine; 4-(2-bromophenyl)butan-2-amine; 4-(2-iodophenyl)butan-2-amine; 4-(2-methoxyphenyl)butan-2-amine; 2-(3-aminobutyl)phenol; 3-(3,4-diethoxyphenyl)propan-1-amine; 4-(4-chlorophenyl)butan-2-amine; 4-(4-methoxyphenyl)butan-2-amine; 2-(5-chloro-1-methyl-1H-indol-3-yl)ethanamine; 1-(5-fluoro-1-methyl-1H-indol-3-yl)propan-2-amine; 1-(5-methoxy-1-methyl-1H-indol-3-yl)propan-2-amine; 2,7-diamino-5-(4-fluorophenyl)-4-oxo-3,4,5,6-tetrahydropyrido[2,3-d]pyrimidine-6-carbonitrile; (Z)-2-(1H-benzo[d]imidazol-2-yl)-N′-hydroxy-3-(4-methoxyphenyl) propanimidamide; a pharmaceutically acceptable salt or solvate, and mixtures thereof.

Embodiment 10: The method of any one of Embodiments 1-9, wherein the subject is further administered at least one additional agent to prevent, treat, and/or ameliorate the cocaine use disorder.

Embodiment 11: The method of any one of Embodiments 1-10, wherein the subject is human.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the present invention. The appended claims are intended to be construed to include all such embodiments and equivalent variations.

1. A method of preventing, treating, and/or ameliorating cocaine use disorder in a subject in need thereof, the method comprising administering to the subject a therapeutically effective amount of at least one dopamine D3 receptor agonist.

2. The method of claim 1, wherein the agonist is selected from the group consisting of:
a compound of formula (I): 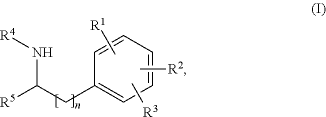
wherein in (I):
R¹, R² and R³ are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and,
n is 2, 3, 4 or 5;
a compound of formula (II): 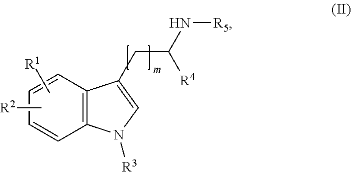
wherein in (II):
R¹ and R² are independently selected from the group consisting of H, cyano, hydroxyl, amino, acetamido, halogen, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R³ and R⁴ are independently selected from the group consisting of H, C₁₋₆ alkyl, and substituted C₁₋₆ alkyl;
R⁵ is selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and,
m is 1, 2, or 3;
2,7-diamino-5-(4-fluorophenyl)-4-oxo-3,4,5,6-tetrahydropyrido[2,3-d]pyrimidine-6-carbonitrile;
(Z)-2-(1H-benzo[d]imidazol-2-yl)-N′-hydroxy-3-(4-methoxyphenyl)propanimidamide;
a pharmaceutically acceptable salt or solvate thereof, and any mixtures thereof.

3. The method of claim 1, wherein the agonist is not addictive to the subject.

4. The method of claim 1, wherein administration of the agonist reduces and/or eliminates the subject's motivation for cocaine use.

5. The method of claim 2, wherein in (I) R¹, R² and R³ are independently selected from the group consisting of H, cyano, halogen, alkoxy, nitro, C₁₋₆ alkyl, and carboxy.

6. The method of claim 2, wherein in (I) R⁴ and R⁵ are independently selected from the group consisting of H, C₁₋₆ alkyl, and substituted C₁₋₆ alkyl.

7. The method of claim 2, wherein in (I) n is 2.

8. The method of claim 2, wherein in (II) m is 1.

9. The method of claim 2, wherein the at least one compound is selected from the group consisting of:
2-amino-4-(2-chlorophenyl)butan-1-ol;
2-(3-aminohexyl)phenol;
4-(2-chlorophenyl)-2-methylamino-butane;
4-(2-chlorophenyl)-2-amino-butane;
4-(2-fluorophenyl)butan-2-amine;
4-(2-bromophenyl)butan-2-amine;
4-(2-iodophenyl)butan-2-amine;
4-(2-methoxyphenyl)butan-2-amine;
2-(3-aminobutyl)phenol;
3-(3,4-diethoxyphenyl)propan-1-amine;
4-(4-chlorophenyl)butan-2-amine;
4-(4-methoxyphenyl)butan-2-amine;
2-(5-chloro-1-methyl-1H-indol-3-yl)ethanamine;
1-(5-fluoro-1-methyl-1H-indol-3-yl)propan-2-amine;
1-(5-methoxy-1-methyl-1H-indol-3-yl)propan-2-amine;
2,7-diamino-5-(4-fluorophenyl)-4-oxo-3,4,5,6-tetrahydropyrido[2,3-d]pyrimidine-6-carbonitrile;
(Z)-2-(1H-benzo[d]imidazol-2-yl)-N′-hydroxy-3-(4-methoxyphenyl)propanimidamide;
a pharmaceutically acceptable salt or solvate thereof, and mixtures thereof.

10. The method of claim 1, wherein the subject is further administered at least one additional agent to prevent, treat, and/or ameliorate the cocaine use disorder.

11. The method of claim 1, wherein the subject is human.
